(19) United States
(12) Patent Application Publication
(10) Pub. No.: US 2004/0268450 A1
(43) Pub. Date: Dec. 30, 2004

(54) Method of producing rose saplings capable of blooming many kinds of roses on each shoot and rose saplings thus produced

(76) Inventor: Tadao Kanda, Kawaguchi-shi (JP)

Correspondence Address: FLYNN THIEL BOUTELL & TANIS, P.C., 2026 RAMBLING ROAD, KALAMAZOO, MI, 49008-1699, US

(21) Appl. No.: 10/869,575

(22) Filed: Jun. 16, 2004

(30) Foreign Application Priority Data
Jun. 27, 2003 (JP) 2003-205065

(51) Int. Cl. A01G 1/00; A01H 5/00

(52) U.S. Cl. 800/323; 47/6; 47/7

(57) ABSTRACT

Commercially available rose saplings have been conventionally grafted a rose scion on a wild rose stock, however shoots grown from a single wild rose rootstock are effectively used to graft many kinds of roses so as to bloom various flowers. The invention provides a method of grafting many kinds of roses on a single wild rose stock to bloom various flowers by paying attention to lateral buds formed on one or several shoots grown from a wild rose rootstock and grafting many kinds of perpetual rose scions on each notched opening with a lateral bud, which makes it possible to enjoy roses planted in a pot even in a small space, and rose saplings thus produced.

### BACKGROUND OF THE INVENTION

1. Field of the Invention

This invention relates to a method of producing rose saplings by grafting many kinds of perpetual gardening roses on each shoot grown from a wild rose rootstock while paying attention to lateral bud regions on a stock having thickness of not less than 1 cm at an end part thereof to use these buds, and rose saplings thus produced, thereby making it possible to bloom roses of different colors, aroma, figures and flower leaves and to enjoy beauty of various roses on a single stock planted in a pot which may be put even in a small space.

Heretofore, commercially available rose saplings have been produced by grafting a scion or a bud on a wild rose stock as shown in FIGS. 4G and 4H.

For that reason, a number of pots and a large space have been necessitated to enjoy beauty of many kinds of roses while they bloom. It is thus difficult to enjoy such various beautiful roses all at once in a small and limited space such as a banquet lobby and a porch of apartment houses. Related Art

In general, it is a conventional manner of raising roses to plant a rose sapling in a pot, grow it and bloom flowers thereof.

In order to enjoy many kinds of roses, a number of pots and thus a considerably large space enough to arrange them are required, which is quite difficult to reserve the space from a standpoint of present housing situation in Japan. As a result, it is restricted to enjoy many kinds of roses including various colors, figures and aroma thereof.

Conventionally, a rose scion has been grafted on a wild rose stock by means of a cut grafting or bud grafting technique to produce a rose sapling, the wild rose being cut off from its rootstock portion and disposed.

### SUMMARY OF THE INVENTION

This invention relates to a method of producing rose saplings by grafting many kinds of perpetual gardening roses on each shoot grown from a wild rose rootstock while paying attention to lateral bud regions on a stock having thickness of not less than 1 cm at an end part thereof to use these buds, thereby making it possible to bloom roses of different colors, aroma, figures and flower leaves and to enjoy beauty of various roses on a single stock planted in a pot which may be put even in a small space.

A method of grafting many kinds of roses on a single wild rose stock to bloom various flowers as means for solving the problem according to the first aspect of the invention comprises employing each shoot grown therefrom as a stock, paying attention to budding vitality of lateral buds between a rootstock and a top end part of the stock to select lateral buds distributed thereon at equal spaces as points to be grafted and grafting thereon different kinds of scions, notches being formed on the stock to graft different kinds of scions, the scions being put therein so that an outer part on a cut scion surface and a notched tongue part having a lateral bud on a stock side are stuck exactly by adjusting cut angles of both parts to securely adhere cambiums thereof on their grafting surface, followed by firm fixture of these parts by means of a vinyl tape to seal from a stock part under a grafted point including the lateral bud to an upper stock-scion junction, thereby controlling germination of the bud, overhead penetration of water or dust and transpiration from the grafted point of the scion and the stock as well as securing vital scion grafting, each scion being grafted in order from the top to the bottom without disturbing a scion just above so as to graft steadily, quickly and effectively.

A rose sapling grafted on a single wild rose stock to bloom many kinds of flowers as means for solving the problem according to the second aspect of the invention produced by employing each shoot grown therefrom as a stock, using lateral buds formed between a rootstock and a top end part of the stock to select lateral buds distributed thereon at equal spaces as points to be grafted and grafting thereon different kinds of scions.

A method of grafting different kinds of roses on a single wild rose stock to bloom various flowers as means for solving the problem according to the third aspect of the invention comprises preparing a stock planted in a pot and roses, as scions, of different kinds and colors planted in pots and grafting each shoot of planted roses on the stock at equal spaces from a top end part to a rootstock thereof, the top end part being shaped into a wedge, while a branched part of scion side being notched to form a grafting point, followed by fixture thereof by means of a vinyl tape to securely adhere cambiums of both stock and scion sides, in which a stem part of the stock is notched to form a groove of about several centimeters as a nearly linear line in a gently slanted situation in the up- to downward direction, the scions are shaved to form a convex angle, both cambiums are stuck securely and further fixed by means of a vinyl tape.

A rose sapling grafted on a single wild rose stock to bloom various flowers as means for solving the problem according to the fourth aspect of the invention produced by preparing a stock planted in a pot and roses, as scions, of different kinds and colors planted in pots to and grafting each shoot of planted roses on the stock at equal spaces from a top end part to a root stock thereof, the top end part being shaped into a wedge, while a branched part of scion side being notched to form a grafting point, followed by fixture thereof by means of a vinyl tape to securely adhere cambiums of both stock and scion sides, in which a stem part of the stock is notched to form a groove of about several centimeters as a nearly linear line in a gently slanted situation in the up- to downward direction, the scions are shaved to form a convex angle, both cambiums are stuck securely and further fixed by means of a vinyl tape.

### BRIEF DESCRIPTION OF THE DRAWINGS

FIG. 1 is a view explaining a method of grafting in which several scions are grafted on a stock having several shoots;

FIG. 2 is a view of explaining a method of grafting in which several scions are grafted on a single stock;

FIG. 3 is view of explaining a method of grafting in which several scions planted in different pots A to D are grafted on a stock;

FIG. 4 is a view showing a manner of grafting wherein FIG. 4G shows a method of grafting a bud is grafted on a stock (bud grafting), FIG. 4H shows a method of grafting scion having a bud on a stock (cut grafting), and FIG. 4K shows a method of grafting a middle portion of nearly linear scion is windingly grafted on a stock;

FIG. 5 is a detailed view in FIGS. 1 and 2 wherein FIG. 5J is a side view of grafting before fixing by means of a vinyl tape, and FIG. 5L is a front view of grafting before fixing by means of a vinyl tape;

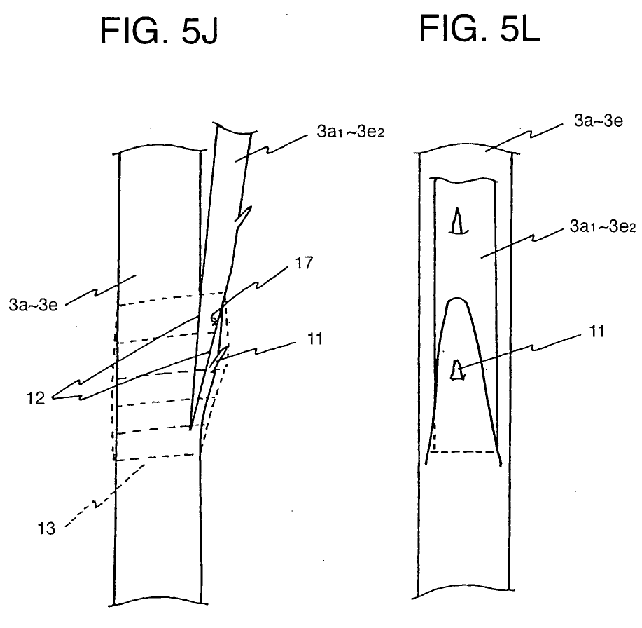

FIG. 6 is a detailed view of FIG. 4K wherein FIG. 6M is a front view of a V-shaped slit, and FIG. 6N is a sectional view taken along line W-W in FIG. 6(M); and

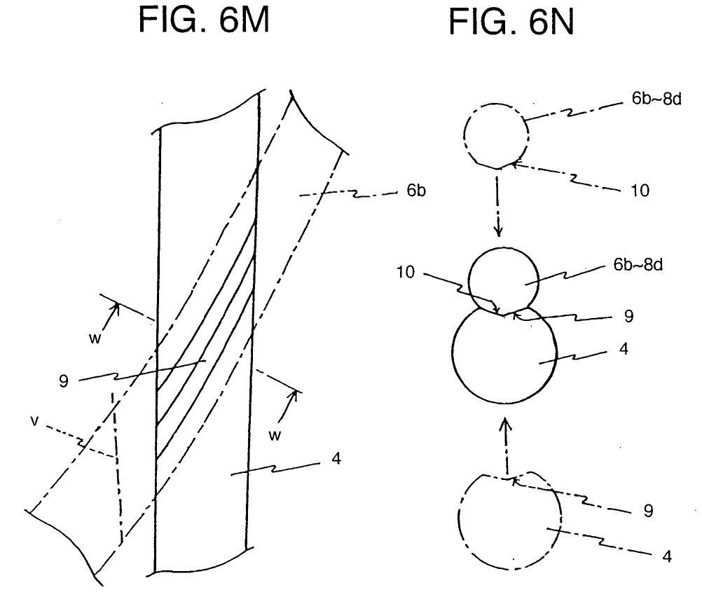

FIG. 7 is a detailed view explaining grafting at a top end part F of stock shown in FIG. 3.

### PREFERRED EMBODIMENTS OF THE INVENTION

Referring now to the drawings, the invention will be described in detail in the following.

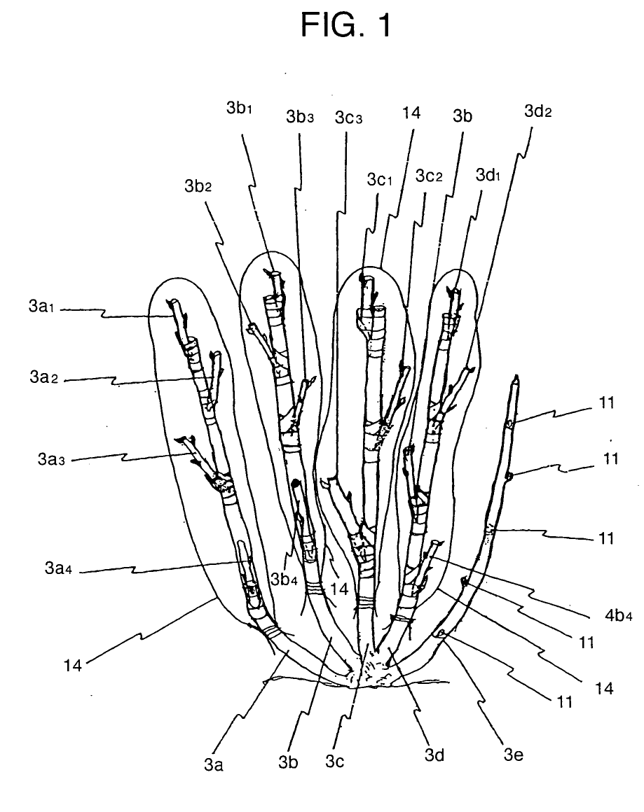

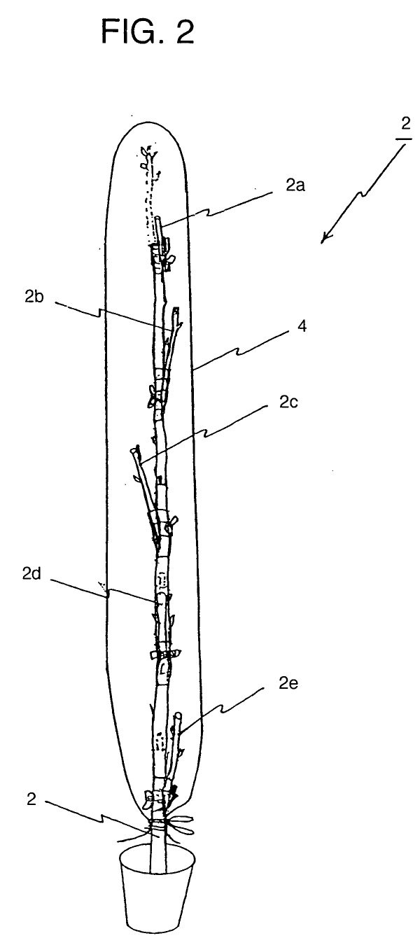

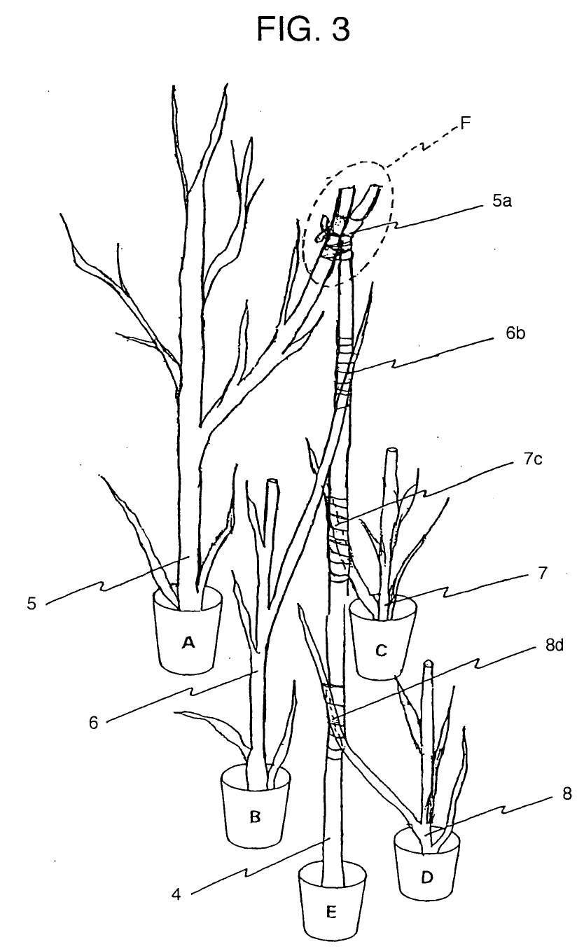

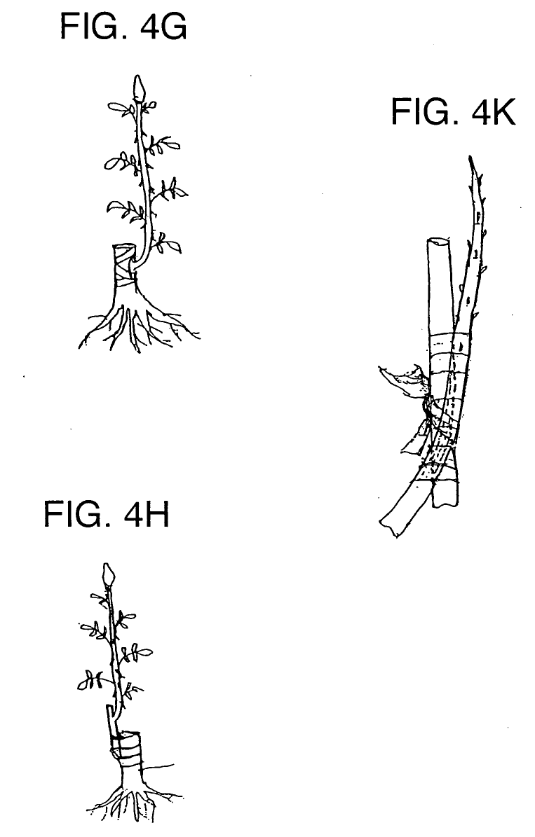

FIG. 1 is a view explaining a method of grafting in which several scions are grafted on each shoot of a stock, while in FIG. 2, they are grafted on a single stock. FIG. 3 is an illustrative view in which several scions planted in different pots are grafted on a stock, while in FIGS. 4G, 4G and 4H are views explaining a manner of grafting.

Working Examples

According to the first aspect of the invention, a various combination of rose scions are grafted on, for example, each shoot of not less than 1 cm in thickness at a top end part thereof, grown from a rootstock of a wild rose, as shown in FIG. 1. The combination may includes different colored perpetual hybrid roses of red 3a₁, white 3a₂, yellow 3a₃ and pink 3a₄; roses of same color 3b having different figures; single flowering roses 3b; single and double flowering roses of the same color 3c; single and double flowering roses of different colors; early- and late-flowering roses; similarly mixed floribunda or polyantha roses; similarly mixed perpetual hybrid tea, floribunda and polyantha roses of same or different colors, and the like. Lateral buds 11 located at equal spaces are selected from those buds dispersed along a stock between the top to the bottom to graft different kinds of roses thereon one by one effectively, as described above. At that time, the lateral bud 11 in the grafting direction is firmly fixed by means of a vinyl tape 13 to prevent pushing out thereof. Such a grafting process is done in order from the top to the bottom to keep out careless touch with a grafted scion just above thereof, thereby the process being proceeded securely, skillfully and effectively.

Then, each grafted shoot is covered and protected respectively with a vinyl bag 14 as a whole to complete the grafting process. As has been described above, this invention is characterized in providing a method of grafting many kinds of roses on a single wild rose stock and rose saplings thus grafted.

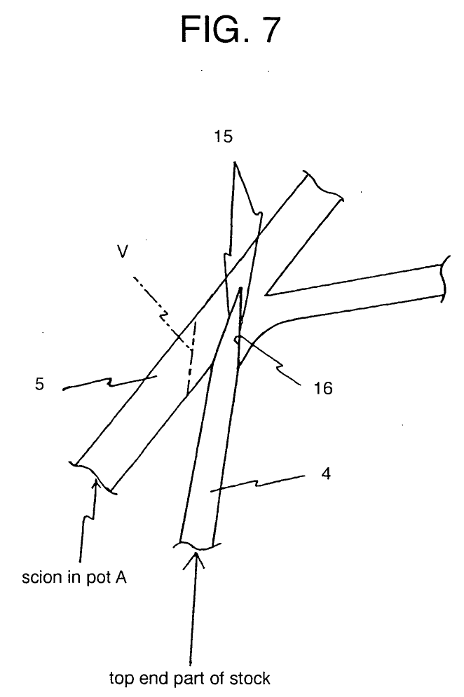

According to the third aspect of the invention, scions 5a to 8d from roses 5 to 8 planted in pots A to D are grafted on a stock 4 at equal spaces from the top to the bottom as a further method of grating as shown in FIG. 3. A grafting manner at the top end is that a branched part of the scion 5 is cut to form a notch 15, while a top end 16 of the stock 4 is cut into a wedge as shown in FIG. 7. The wedged stock is then firmly inserted into or put on the notch 15 to adhere both cambiums thereof each other, followed by fixing by winding a vinyl tape therearound. The thus adhered cambiums are allowed to stand for a while and, after a sprout comes out and a grafted condition is fixed, a circular peeling treatment is done and then the scion is cut off at a line V depending on a condition thereof to complete the top end part. According to the invention, a method of grafting of scions 6b to 8d on a stem 4 of a stock is that the stock 4 is notched to form a groove 9 of about 3 cm as a nearly linear line in a gently slanted situation K in the up- to downward direction, while the scions 6b to 8d are shaved to form a convex angle 10, so that both cambiums thereof are stuck securely and further fixed by means of a vinyl tape. The thus adhered cambiums are allowed to stand for a while and, after a sprout comes out and a grafted condition is fixed, a circular peeling treatment is done and then the scion is cut off at a line V depending on a condition thereof to complete a rose sapling capable of blooming many kinds of roses on a single stock.

There have been produced roses of standard type, which bloom on the top of a wild rose stem as a stock of about 1 meter high, for the purpose of enjoying flowers at a level of eyes and preventing diseases caused by splashed mud due to rain, etc. Only one kind of rose is grafted on one point to bloom at a higher position than usual.

According to the invention, it is possible to bloom various flowers on a single wild rose stock planted in a pot by grafting many kinds of roses one after another on each shoot grown therefrom having thickness of not less than 1 cm at a top end part thereof to use these buds. The invention successfully provides rose saplings which bloom various roses even in a limited space such as porches, terrace and lobbies and brings joy to many people effectively.

According to the invention, lateral buds to be grown as stems of a wild rose stock are basically left, while already grown shoots are removed.

A grafted stock is covered with a vinyl bag 14 to control dry out thereof.

The vinyl bag 14 is then removed after sprout out of new buds and their vigorous growth are observed.

Rose saplings which bloom various flowers on shoots as stocks grown from a wild rose stock root are produced completely in a manner as described above.

According to conventional methods for producing grafted rose saplings, shoots of wild roses as a stock are cut off from their rootstocks and disposed. In order to make effective use of them without cutting off, there have been successfully tried a method of grafting several kinds of rose scions on a single stock as shown in FIGS. 2 and 3, and a further method of grafting similar roses on each shoot grown from a wild rose rootstock, which has thickness of not less than 1 cm at an end part thereof, as shown in FIG. 1.

A vital grafting rate of scions is improved by selecting location of wild rose buds on the stock and making use of a water raising action for vigorous sprouting. Scions are grafted on buds of the stock at equal spaces in order from the top to the bottom while changing a grafting point, thereby grafting operation being done steadily, quickly and effectively without disturbing a scion grafted just above.

Roses are enjoyed in pots or in gardens. In a case of potted one, a rose is planted and raised in a pot to bloom flowers as a quite general cultivating manner. As a number of pots should be used to enjoy lots of roses, a considerably large space is required but is rather difficult to reserve such a space from a standpoint of present housing situation in Japan. As a result, enjoyment of rose of various kinds such as different colors, figures and aroma is restricted self-evidently.

The invention is based on a greedy idea to effectively give pleasure of roses to many people even in a limited space such as porches, terraces and lobbies and successfully provides rose saplings capable of blooming various flowers produced by grafting many kinds of roses one after another on each shoots grown from a single wild rose stock planted in a port and having thickness of not less than 1 cm at an end part thereof.

What is claimed is:

1. A method of grafting many kinds of roses on a single wild rose stock to bloom various flowers as means for solving the problem according to the first aspect of the invention comprises employing each shoot grown therefrom as a stock, paying attention to budding vitality of lateral buds between a rootstock and a top end part of the stock to select lateral buds distributed thereon at equal spaces as points to be grafted and grafting thereon different kinds of scions, notches being formed on the stock to graft different kinds of scions, the scions being put therein so that an outer part on a cut scion surface and a notched tongue part having a lateral bud on a stock side are stuck exactly by adjusting cut angles of both parts to securely adhere cambiums thereof on their grafting surface, followed by firm fixture of these parts by means of a vinyl tape to seal from a stock part under a grafted point including the lateral bud to an upper stock-scion junction, thereby controlling germination of the bud, overhead penetration of water or dust and transpiration from the grafted point of the scion and the stock as well as securing vital scion grafting, each scion being grafted in order from the top to the bottom without disturbing a scion just above so as to graft steadily, quickly and effectively.

2. A rose sapling grafted on a single wild rose stock to bloom many kinds of flowers as means for solving the problem according to the second aspect of the invention produced by employing each shoot grown therefrom as a stock, using lateral buds formed between a rootstock and a top end part of the stock to select lateral buds distributed thereon at equal spaces as points to be grafted and grafting thereon different kinds of scions.

3. A method of grafting different kinds of roses on a single wild rose stock to bloom various flowers as means for solving the problem according to the third aspect of the invention comprises preparing a stock planted in a pot and roses, as scions, of different kinds and colors planted in pots and grafting each shoot of planted roses on the stock at equal spaces from a top end part to a rootstock thereof, the top end part being shaped into a wedge, while a branched part of scion side being notched to form a grafting point, followed by fixture thereof by means of a vinyl tape to securely adhere cambiums of both stock and scion sides, in which a stem part of the stock is notched to form a groove of about several centimeters as a nearly linear line in a gently slanted situation in the up- to downward direction, the scions are shaved to form a convex angle, both cambiums are stuck securely and further fixed by means of a vinyl tape.

4. A rose sapling grafted on a single wild rose stock to bloom various flowers as means for solving the problem according to the fourth aspect of the invention produced by preparing a stock planted in a pot and roses, as scions, of different kinds and colors planted in pots to and grafting each shoot of planted roses on the stock at equal spaces from a top end part to a root stock thereof, the top end part being shaped into a wedge, while a branched part of scion side being notched to form a grafting point, followed by fixture thereof by means of a vinyl tape to securely adhere cambiums of both stock and scion sides, in which a stem part of the stock is notched to form a groove of about several centimeters as a nearly linear line in a gently slanted situation in the up- to downward direction, the scions are shaved to form a convex angle, both cambiums are stuck securely and further fixed by means of a vinyl tape.
